(19) United States
(12) Patent Application Publication
(10) Pub. No.: US 2014/0148330 A1
(43) Pub. Date: May 29, 2014

(54) SEMI-PERMEABLE PARTICLES HAVING METALLIC CATALYSTS AND USES

(71) Applicants: Douglas R. Robello, Webster, NY (US); Mridula Nair, Penfield, NY (US); Mark R. Mis, Hornell, NY (US); Matthew Dirmyer, Rochester, NY (US)

(72) Inventors: Douglas R. Robello, Webster, NY (US); Mridula Nair, Penfield, NY (US); Mark R. Mis, Hornell, NY (US); Matthew Dirmyer, Rochester, NY (US)

(21) Appl. No.: 13/686,941

(22) Filed: Nov. 28, 2012

(51) Int. Cl. B01J 31/06; C07C 67/303; C07C 29/17

(52) U.S. Cl. CPC B01J 31/06; C07C 29/17; C07C 67/303
USPC 502/159; 568/903; 560/263

(57) ABSTRACT

Semi-permeable particle can be used to facilitate chemical reactions. The semi-permeable particles are permeable to molecules having a molar mass of 1000 Daltons or less, have a mode particle size of at least 1 μm, and comprise nanoparticles of catalytically active metallic materials disposed within at least some of multiple discrete cavities in the continuous polymeric phase. The nanoparticles of catalytically active metallic materials (a) comprise one or more elements selected from Groups 8, 9, 10, and 11 of the Periodic Table, and (b) have an effective diameter of at least 1 nm and up to and including 200 nm.

### COPENDING APPLICATIONS

Reference is made to copending and commonly assigned U.S. Ser. No. 13/______ (filed on even date herewith by Mis, Nair, and Robello and entitled PARTICLES CONTAINING ORGANIC CATALYTIC MATERIALS AND USES, Attorney Docket No. K001099/MT).

Reference is made to copending and commonly assigned U.S. Ser. No. 13/______ (filed on even date herewith by Nair and Jones and entitled POROUS ORGANIC POLYMERIC FILMS AND PREPARATION, Attorney Docket No. K001040/JLT).

Reference is made to copending and commonly assigned U.S. Ser. No. 13/______ (filed on even date herewith by Nair, Jones, and Mis and entitled POROUS PARTICLES AND METHODS OF MAKING THEM, Attorney Docket No. K001046/JLT).

### FIELD OF THE INVENTION

This invention relates to micro-sized semi-permeable particles in which catalytic materials, such as catalytically active metallic materials, have been incorporated. This invention also relates to methods of making and using these semi-permeable particles.

### BACKGROUND OF THE INVENTION

Porous polymeric particles have been prepared and used for many different purposes. For example, porous particles have been described for use in chromatographic columns, ion exchange and adsorption resins, cosmetic formulations, papers, and paints. The methods for generating pores in polymeric particles are well known in the field of polymer science. However, each particular porous particle often requires unique methods for their manufacture. Some methods of manufacture produce large particles without any ability to control of the pore size while other manufacturing methods control the pore size without controlling the overall particle size.

Marker materials can be included in porous particles so that the particles can be detected for a specific purpose. For example, U.S. Patent Applications 2008/0176157 (Nair et al.) and 2010/0021838 (Putnam et al.) and U.S. Pat. No. 7,754,409 (Nair et al.) describe porous particles and a method for their manufacture, which porous particles are designed to be toner particles for use in electrophotography. Such porous particles typically contain a colorant and can be prepared using a multiple emulsion process in combination with a suspension process (such as “evaporative limited coalescence”, ELC) in a reproducible manner and with a narrow particle size distribution.

Still another important use of polymeric particles is as a means for marking documents, clothing, or labels as a “security” tag, for example for authentication of documents using an electrophotographic process and core-shell toner particles containing an infrared emitting component and a detection step. For example, U.S. Patent Application Publication 2003/0002029 (Dukler et al.) describes a method for labeling documents for authentication using a toner particle containing two or more mixed compounds having a characteristic detectable signal.

U.S. Pat. No. 8,110,628 (Nair et al.) describes porous particles and articles containing same that contain various marker materials within discrete pores for specific means of detection. These porous particles can be prepared using multiple water-in-oil emulsions containing the desired markers and pore stabilizing hydrocolloids to prevent coalescence of the pore forming water-in-oil droplets.

Catalytic metal nanoparticles encapsulation in microcapsules is described by Parthasarathy et al. in J. Applied Polymer Sci., 62, 875-886 (1996). However, these microcapsules are tubular and do not contain multiple discrete cavities.

Chemically reactive materials can be used in compositions for many purposes but there is always a need to protect people and the environment from chemicals such as catalysts used in chemical reactions. There is also a desire for a way to have easier handling of catalytically active metallic nanoparticulate materials. There is a further desire to find a means for providing micro-sized reactive materials containing reactive nano-sized materials that can also be reused while retaining high reactive capability.

### SUMMARY OF THE INVENTION

The present invention provides a semi-permeable particle comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the discrete cavities, the semi-permeable particle being permeable to molecules having a molar mass of 1000 Daltons or less,

wherein the semi-permeable particle has a mode particle size of at least 1 μm and comprises nanoparticles of catalytically active metallic materials disposed within at least some of the multiple discrete cavities,

which nanoparticles of catalytically active metallic materials (a) comprise one or more elements selected from Groups 8, 9, 10, and 11 of the Periodic Table, and (b) have an effective diameter of at least 1 nm and up to and including 200 nm.

An aqueous slurry of multiple semi-permeable particles according to any embodiments of the present invention can also be obtained using the present invention.

In addition, the present invention provides a method of making an aqueous dispersion of a plurality of semi-permeable particles, each semi-permeable particle further comprising multiple discrete cavities within the continuous polymeric phase, and a cavity stabilizing hydrocolloid disposed within at least some of the multiple discrete cavities, the semi-permeable particle being permeable to molecules having a molar mass of 1000 Daltons or less,

wherein the semi-permeable particle has a mode particle size of at least 1 μm and comprises nanoparticles of catalytically active metallic materials disposed within at least some of the multiple discrete cavities,

which nanoparticles of catalytically active metallic materials (a) comprise one or more elements selected from Groups 8, 9, 10, and 11 of the Periodic Table, and (b) have an effective diameter of at least 1 nm and up to and including 200 nm,

the method comprising:

providing a first aqueous phase comprising the nanoparticles of catalytically active metallic materials and the cavity stabilizing hydrocolloid, both dispersed within the first aqueous phase,
dispersing the first aqueous phase in an organic solvent comprising the water-insoluble semi-permeable polymer to form a first water-in-oil emulsion,
dispersing the first water-in-oil emulsion in a second aqueous phase containing a surface stabilizing material to form a water-in-oil-in-water emulsion containing droplets of the water-in-oil emulsion, and
removing the organic solvent from the droplets to form the aqueous dispersion of a plurality of semi-permeable particles.

Further, this invention provides a method for causing a chemical reaction, comprising:

contacting one or more reactive chemicals having a molar mass of 1000 Daltons or less with a slurry of semi-permeable particles,

each of the semi-permeable particles comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the multiple discrete cavities, the semi-permeable particle being permeable to one or more reactive chemicals having a molar mass of 1000 Daltons or less,

wherein the semi-permeable particle has a mode particle size of at least 1 μm and comprises nanoparticles of catalytically active metallic materials disposed within at least some of the multiple discrete cavities, the catalytically active metallic materials capable of catalyzing a chemical conversion of the one or more reactive chemicals having a molar mass of 1000 Daltons or less,

which nanoparticles of catalytically active metallic materials (a) comprise one or more elements selected from Groups 8, 9, 10, and 11 of the Periodic Table, and (b) have an effective diameter of at least 1 nm and up to and including 200 nm.

The present invention provides a number of advantages. The catalytically active metallic materials (as nanoparticles) having elements from one or more of Groups 8-11 of the Periodic Table, which are used in the present invention can be very expensive and difficult to isolate for recovery and reuse. The relatively large semi-permeable particles in which they are incorporated for this invention allow for their recovery by simple filtration or centrifugation. Little or no nanoparticles are lost from the semi-permeable particles when they are recovered and this provides considerable economic advantages.

The semi-permeable particles of this invention have multiple discreet cavities that allow diffusion of the intended small molecule reactive chemicals so that they interact with the catalytically active metallic materials present in the multiple discrete cavities. This provides desired rapid reaction rates while allowing convenient isolation of those catalytically active metallic materials after use.

Similarly, isolation of a reaction product obtained using the catalytically active metallic materials contained in the semi-permeable polymer particles, is facilitated so that contamination is minimized. In principle, any catalytically active metallic material can be used in the present invention if it can be dispersed in water. The uniform size of the semi-permeable particles of this invention leads to consistent reaction rates and recovery processes. In addition, the semi-permeable particles of the invention need not be isolated or dried before they are used. Rather, they can be used in an aqueous slurry that is obtained after removal of solvent.

### DETAILED DESCRIPTION OF THE INVENTION

### Definitions

As used herein to define various components of solutions, formulations, and components, unless otherwise indicated, the singular forms “a”, “an”, and “the” are intended to include one or more of the components (that is, including plurality referents).

Each term that is not explicitly defined in the present application is to be understood to have a meaning that is commonly accepted by those skilled in the art. If the construction of a term would render it meaningless or essentially meaningless in its context, the term's definition should be taken from a standard dictionary.

The use of numerical values in the various ranges specified herein, unless otherwise expressly indicated otherwise, are considered to be approximations as though the minimum and maximum values within the stated ranges were both preceded by the word “about”. In this manner, slight variations above and below the stated ranges can be used to achieve substantially the same results as the values within the ranges. In addition, the disclosure of these ranges is intended as a continuous range including every value between the minimum and maximum values.

The terms “semi-permeable particle” and “semi-permeable porous particles” are used herein, unless otherwise indicated, to refer to materials of the present invention. They are defined in more detail below.

The term “porogen” refers to a cavity forming agent used to make the semi-permeable particles. In this invention, a porogen can be the first aqueous phase of the water-in-oil emulsions, the cavity stabilizing hydrocolloid, or any other additive in the aqueous phase that can modulate the porosity of the semi-permeable particles.

In this invention, the term “discrete cavity” is used instead of “pore” to define a void within the continuous polymeric phase of the semi-permeable particles. Multiple discrete cavities can be interconnected to form a network of voids or they can exist in isolation from other discrete cavities.

The semi-permeable particles can include “micro”, “meso”, and “macro” discrete cavities, which according to the International Union of Pure and Applied Chemistry, are the classifications recommended for discrete cavities less than 2 nm, from 2 nm to 50 nm, and greater than 50 nm, respectively. The semi-permeable particles can include closed discrete cavities of all sizes and shapes (cavities entirely within the continuous polymeric phase). While there may be open cavities on the surface of the semi-permeable particle, such open cavities are not desirable and are generally present only by accident. The size of the semi-permeable particle, the formulation, and manufacturing conditions are the primary controlling factors for discrete cavity size.

The multiple discrete cavities can have an average size of at least 100 nm and up to and including 5 μm or typically at least 500 nm and up to and including 3 μm. For spherical discrete cavities, this average size is an average diameter. For non-spherical discrete cavities, the average size refers to the average largest dimension”. The discrete cavities can have the same or different average sizes. Discrete cavity size can be determined by analyzing Scanning Electron Microscopy (SEM) images of fractured semi-permeable particles using a commercial statistical analysis software package. For example, the average discrete cavity size can be determined by calculating the average diameter of 20 measured discrete cavities.

### Uses

The semi-permeable particles of this invention can have various uses including but not limited to use in drug delivery devices, cosmetic formulations, pharmaceuticals and diagnostic and analytical devices, and chemical reactors used for organic syntheses or other chemical processes, food processing, laundering, waste water treatment, air pollution abatement, bio fuels refining, fuel cells, convertors, or any applications where a catalytically active metallic material is needed for a chemical reaction.

### Semi-Permeable Particles

The semi-permeable particles comprise a continuous polymeric phase formed from one or more water-insoluble semi-permeable polymers (defined below) including an external particle surface and multiple discrete cavities dispersed within the continuous polymeric phase and nanoparticles of catalytically active metallic materials (defined below) that are primarily within the multiple discrete cavities.

In most embodiments, the continuous polymeric phase of the semi-permeable particles has the same composition. That is, the continuous polymeric phase is uniform in composition including any additives that may be incorporated into the water-insoluble semi-permeable polymer. In addition, if mixtures of water-insoluble semi-permeable polymers are used in the continuous polymeric phase, those mixtures are dispersed uniformly throughout.

The semi-permeable particles are generally prepared, as described below, using multiple water-in-oil emulsions in combination with an aqueous suspension process, such as in the ELC process.

The water-insoluble semi-permeable polymers useful in the practice of this invention to provide the continuous polymeric phase can be any type of polymer or resin that is capable of being dissolved in a suitable solvent (described below) and is insoluble in water. In addition, these water-insoluble polymers are “semi-permeable”, meaning that relatively large catalytically active metallic materials (nanoparticles, for example, having a diameter greater than 1 nm) are unable to penetrate the continuous polymeric phase that makes up the walls of the multiple discrete cavities and are therefore retained indefinitely, while smaller reactants and products can freely diffuse through the discrete cavity walls and the continuous polymeric phase.

Useful water-insoluble semi-permeable polymers include but are not limited to, those derived from vinyl monomers such as styrene monomers and condensation monomers such as esters and mixtures thereof. Such polymers include but are not limited to, homopolymers and copolymers such as polyesters, styrenic polymers (for example polystyrene and polychlorostyrene), mono-olefin polymers (for example, polymers formed from one or more of ethylene, propylene, butylene, and isoprene), vinyl ester polymers (for example, polymer formed from one or more of vinyl acetate, vinyl propionate, vinyl benzoate, and vinyl butyrate), acrylic polymers for example formed from one or more α-methylene aliphatic monocarboxylic acid esters (for example, polymers formed from one or more of methyl acrylate, ethyl acrylate, butyl acrylate, dodecyl acrylate, octyl acrylate, phenyl acrylate, methyl methacrylate, ethyl methacrylate, butyl methacrylate, and dodecyl methacrylate), vinyl ether polymers (such as polymers formed from one or more of vinyl methyl ether, vinyl ethyl ether, and vinyl butyl ether), vinyl ketone polymers (for example, polymers formed from one or more of vinyl methyl ketone, vinyl hexyl ketone, and vinyl isopropenyl ketone), and aliphatic cellulose ester polymers. Particularly useful water-insoluble, semi-permeable polymers include polystyrenes (including homopolymers and copolymers of styrene derivatives), polyesters, styrene/alkyl acrylate copolymers, styrene/alkyl methacrylate copolymers, styrene/acrylonitrile copolymers, styrene/butadiene copolymers, styrene/maleic anhydride copolymers, polyethylene resins, and polypropylene resins. Other useful water-insoluble semi-permeable polymers include polyurethanes, urethane acrylic copolymers, epoxy resins, silicone resins, polyamide resins, modified rosins, paraffins, and waxes. Still other useful water-insoluble semi-permeable polymers are polyesters of aromatic or aliphatic dicarboxylic acids with one or more aliphatic diols, such as polyesters of isophthalic or terephthalic or fumaric acid with diols such as ethylene glycol, cyclohexane dimethanol, and bisphenol adducts of ethylene or propylene oxides. The polyesters can be saturated or unsaturated.

Particularly useful water-insoluble, semi-permeable polymers are selected from polyesters, polyamides, polyurethanes, styrenic polymers, mono-olefin polymers, vinyl ester polymers, acrylic polymers, vinyl ether polymers, vinyl ketone polymers, and aliphatic cellulose ester polymers.

One or more cavity stabilizing hydrocolloids are disposed within at least some of the multiple discrete cavities, and typically, these compounds are disposed within essentially all (at least 95%) of the multiple discrete cavities. Suitable cavity stabilizing hydrocolloids include but are not limited to, both naturally occurring and synthetic, water-soluble or water-swellable polymers selected from the group consisting of cellulose derivatives [such as for example, carboxymethyl cellulose (CMC) that is also referred to as sodium carboxymethyl cellulose], gelatin (for example, alkali-treated gelatin such as cattle bone or hide gelatin, or acid treated gelatin such as pigskin gelatin), gelatin derivatives (for example, acetylated gelatin and phthalated gelatin), proteins and protein derivatives, hydrophilic synthetic polymers [such as poly(vinyl alcohol), poly(vinyl lactams), acrylamide polymers, polyvinyl acetals, polymers of alkyl and sulfoalkyl acrylates and methacrylates, hydrolyzed polyvinyl acetates, polyamides, polyvinyl pyridine, and methacrylamide copolymers], water soluble microgels, polyelectrolytes [such as a polystyrene sulfonate, poly(2-acrylamido-2-methylpropanesulfonate), and a polyphosphate], and mixtures of any of these classes of materials.

In order to stabilize the initial water-in-oil emulsions so that they can be held without ripening or coalescence, it is desired that the cavity stabilizing hydrocolloids in the aqueous phase have a higher osmotic pressure than that of the oil phase depending on the solubility of water in the oil. This reduces the diffusion of water into the oil phase from the aqueous phases and thus reduces the ripening caused by migration of water between the water droplets. One can achieve a higher osmotic pressure in the aqueous phase either by increasing the concentration of the cavity stabilizing hydrocolloid or by increasing the charge on the cavity stabilizing hydrocolloid (the counter-ions of the dissociated charges on the cavity stabilizing hydrocolloid increase its osmotic pressure). It can be advantageous to have weak base or weak acid moieties in the cavity stabilizing hydrocolloids that allow for their osmotic pressures to be controlled by changing the pH. Such cavity stabilizing hydrocolloids are considered “weakly dissociating hydrocolloids”. For these weakly dissociating hydrocolloids, the osmotic pressure can be increased by buffering the pH to favor dissociation, or by simply adding a base (or acid) to change the pH of the aqueous phase to favor dissociation. One example of such a weakly dissociating hydrocolloid is CMC that has a pH sensitive dissociation (the carboxylate is a weak acid moiety). For CMC, the osmotic pressure can be increased by buffering the pH, for example using a pH 6-8 buffer, or by simply adding a base to raise the pH of the aqueous phase to favor dissociation. For aqueous phases containing CMC, the osmotic pressure increases rapidly as the pH is increased from 4-8.

Other synthetic polyelectrolyte hydrocolloids such as polystyrene sulfonate (PSS), poly(2-acrylamido-2-methylpropanesulfonate) (PAMS), and polyphosphates are also useful cavity stabilizing hydrocolloids.

Particularly useful cavity stabilizing hydrocolloids are selected from the group consisting of carboxymethyl cellulose (CMC), a gelatin, a protein or protein derivative, a hydrophilic synthetic polymer, a water-soluble microgel, a polystyrene sulfonate, poly(2-acrylamido-2-methylpropanesulfonate), and a polyphosphate.

The cavity stabilizing hydrocolloids are soluble in water and have no negative impact on multiple emulsification processes, or the catalytically active metallic materials. The cavity stabilizing compounds can be optionally crosslinked to minimize migration of the cavity stabilizing hydrocolloid from the discrete cavities.

The amount of the one or more cavity stabilizing hydrocolloids in the semi-permeable particles will depend on the amount of porosity and size of the multiple discrete cavities desired and the molecular weight and charge of the cavity stabilizing hydrocolloid that is chosen. For example, the one or more cavity stabilizing hydrocolloids can be present in the semi-permeable particles in an amount of at least 0.5 weight % and up to and including 20 weight %, or typically at least 1 weight % and up to and including 10 weight %, based on total semi-permeable particle dry weight.

To provide additional stability of multiple discrete cavities in the water-in-oil emulsions and resulting semi-permeable particles, the oil phase can also comprise low HLB polymeric emulsifiers preferably, one or more amphiphilic (low HLB) block copolymers (emulsifiers) that are disposed at the interface of the multiple discrete cavities and the continuous polymeric solid phase of the semi-permeable particles. The term “amphiphilic” is generally used to refer to a molecule having a polar, water-soluble group that is attached to a non-polar, water-insoluble hydrocarbon or oleophilic group. “HLB” refers to the well known term “hydrophilic-lipophilic balance” and refers to the measure of the degree to which a compound is hydrophilic or lipophilic and is determined for a given polymer or molecule using the known Griffin's mathematical method where HLB equals 20 (Mₕ/M) wherein Mₕ equals the molecular weight of the hydrophilic block in the molecule and M equals the molecular weight of the whole block copolymer. Thus, the amphiphilic block copolymers useful in the present invention have a low HLB value, meaning that they are more lipophilic than hydrophilic, and they comprise both water-soluble blocks (hydrophilic) and water-insoluble blocks (lipophilic), and the HLB value is less than or equal to 6.

The molecular weights of the water-soluble component and the oleophilic components are not critical as long as the resulting amphiphilic block copolymer has an HLB equal to or less than 6. For example, the block copolymers can have a hydrophilic block having a molecular weight (Mₕ) of at least 100 and up to and including 25,000, and a hydrophobic (or oleophilic) block having a molecule weight (Mₙ) of at least 500 to and including 100,000.

In some embodiments, the amphiphilic block copolymer comprises a hydrophilic segment comprising polyethyleneoxide and a hydrophobic (oleophilic) segment comprising polycaprolactone. Further details of such block copolymers are provided in Kowalski et al., Macromol. Rapid Commun., 1998, Vol. 19, 567, and in U.S. Pat. No. 5,429,826 (Nair et al.) that is incorporated herein by reference.

Other useful hydrophilic components for amphiphilic block copolymers can be derived from poly(2-ethyloxazolines), poly(saccharides), and dextrans.

The oleophilic block component of the amphiphilic block copolymers useful in the present invention can also be selected from many common components, including but not limited to, oleophilic components derived from monomers such as: styrene, caprolactone, propiolactone, β-butyrolactone, δ-valerolactone, c-caprolactam, lactic acid, glycolic acid, hydroxybutyric acid, and derivatives of lysine and glutamic acid. Particularly useful oleophilic components of the amphiphilic block copolymers useful in this invention are derived from polymers such as certain polyesters, polycarbonates, and polyamides, or more particularly polyesters such as poly(caprolactone) and its derivatives, poly(lactic acid), poly(3-hydroxybutyrate-co-3-hydroxyvalerate), poly(3-hydroxybutyrate), and poly(glycolic acid).

A particularly useful amphiphilic block copolymer can be defined as an A-B block copolymer that comprises a hydrophilic block (A) comprising polyethyleneoxide and a hydrophobic (oleophilic) block (B) comprising polycaprolactone represented herein as PEO-b-PCL.

The amphiphilic block copolymers can also be represented as “A-B-A” type wherein A and B are defined above. Although this invention is directed mainly towards amphiphilic block copolymers, graft copolymers and random graft copolymers containing similar components are also useful.

The amphiphilic block copolymer can be present in the resulting semi-permeable particles in an amount of at least 1 weight % and up to and including 99.5 weight %, or at least 2 weight % and up to and including 50 weight %, based on total semi-permeable particle weight. It is contemplated that in some embodiments, the amphiphilic block copolymer can comprise the continuous polymeric solid phase of the semi-permeable particles and at the same time, function as the low HLB material that is disposed at the interface of the multiple discrete cavities.

In the method of this invention, the amphiphilic block copolymer can be present in the oil phase in an amount of at least 0.2 weight % and up to and including 30 weight %, or typically of at least 0.5 weight % and up to and including 15 weight %, based on the total oil phase weight.

While low HLB amphiphilic block copolymers are preferred as the optional emulsifiers for preparing the water-in-oil emulsions, other polymeric emulsifiers are also envisioned as useful depending on the composition of the oil phase. An example of such an emulsifier is GRINDSTED® PGPR 90, polyglycerol polyricinolate emulsifier, obtained from Dupont.

The semi-permeable particles of this invention are permeable to molecules having a molar mass of 1000 Daltons or less (1000 or less molecular weight, or 1000 g or less per mole of the molecule). In this context, the term “permeable” refers to the ability of a molecule to penetrate the continuous polymeric phase (that composes the walls of the multiple discrete cavities) at a useful rate. Such molecules to which the semi-permeable particles are permeable include but are not limited to, organic molecules that are partially or completely soluble in water.

The semi-permeable particles of this invention generally have a mode particle size of at least 1 μm and up to and including 100 μm, or typically of at least 4 μm and up to an including 50 μm. This mode particle size can be measured by automated image analysis and flow cytometry using any suitable equipment designed for that purpose. The mode particle size represents the most frequently occurring diameter for spherical semi-permeable particles and the largest diameter for the non-spherical semi-permeable particles.

In general, the volume of the multiple discrete cavities in the semi-permeable particles is at least 10% and up to and including 60%, or more likely at least 20% and up to and including 50% based on the total dry semi-permeable particle volume. This porosity can be measured by the mercury intrusion technique.

The semi-permeable particles of this invention can be spherical or non-spherical depending upon the desired use. The shape of semi-permeable particles can be characterized by an “aspect ratio” that is defined as the ratio of the largest perpendicular length to the longest length of the semi-permeable particle. These lengths can be determined for example by optical measurements using a commercial particle shape analyzer such as the Sysmex FPIA-3000 (Malvern Instruments). For example, semi-permeable particles that are considered “spherical” for this invention can have an aspect ratio of at least 0.95 and up to and including 1. For the non-spherical semi-permeable particles of this invention, the aspect ratio can be at least 0.4 and up to and including 0.95.

The semi-permeable particles comprise one or more types of nanoparticles of catalytically active metallic materials that are disposed within at least some of the multiple discrete cavities, and usually within at least 80% and up to and including 100% of the multiple discrete cavities. These nanoparticles of catalytically active metallic materials comprise one or more elements selected from one or more of Groups 8, 9, 10, and 11 of the Periodic Table, including but not limited to, iron, cobalt, nickel, copper, ruthenium, palladium, rhodium, silver, osmium, iridium, platinum, and gold. Nanoparticles of catalytically active metallic materials comprising palladium, platinum, rhodium, ruthenium, nickel, cobalt, iron, copper, silver, gold, iridium, and osmium are particularly useful in the discrete cavities, and nanoparticles of catalytically active metallic materials comprising palladium, platinum, or nickel are most useful. The catalytically active metallic materials can comprise the described metallic elements as well as compounds comprising the metal elements such as metal alloys (such as an alloy of copper and chromium), metal oxides (such as platinum oxide, osmium oxide, and iron oxide), and metal sulfides (such as nickel sulfide and iron sulfide). In some embodiments, the aqueous slurry of multiple semi-permeable particles of this invention comprise one or more nanoparticles of catalytically active metallic materials comprising palladium, platinum, rhodium, ruthenium, nickel, cobalt, iron, copper, silver, gold, iridium, and osmium.

The catalytically active metallic materials generally have an effective diameter of at least 1 nm and up to and including 200 nm, typically at least 2 nm and up to and including 100 nm, or at least 2 nm and up to and including 50 nm. These dimensions are meant to define the term “nanoparticles”.

In some embodiments, the semi-permeable particles of this invention comprise nanoparticles of catalytically active metallic materials comprising palladium, platinum, or nickel in the discrete cavities, the nanoparticles having an effective diameter of at least 2 nm and up to and including 100 nm.

The semi-permeable particles of this invention can also comprise one or more surface stabilizing materials on the external particle surface of each particle. Useful surface stabilizing materials include but are not limited to, stabilizer polymers such as poly(vinyl pyrrolidone) and poly(vinyl alcohol), inorganic stabilizers such as clay particles, colloidal or fumed silica (for example LUDOX™ or NALCO™), or polymer latex particles as described in modified ELC process described in U.S. Pat. No. 4,833,060 (Nair et al.), U.S. Pat. No. 4,965,131 (Nair et al.), U.S. Pat. No. 2,934,530 (Ballast et al.), U.S. Pat. No. 3,615,972 (Morehouse et al.), U.S. Pat. No. 2,932,629 (Wiley), and U.S. Pat. No. 4,314,932 (Wakimoto et al.), the disclosures of which are hereby incorporated by reference. Any combinations of these surface stabilizing materials can also be used.

The actual amount of surface stabilizing material present on the semi-permeable particles depends on the size of the semi-permeable particles desired, which in turn depends upon the volume and weight ratios of the various phases used for making the emulsions (described below). While not intending to be limiting for this invention, the amount of surface stabilizing material on the semi-permeable particles can be at least 0.5 weight % and up to and including 30 weight %, or typically at least 2 weight % and up to and including 20 weight %, based on the total dry weight of the semi-permeable particles and depending upon the particle size of the surface stabilizing material (for example, colloidal or fumed silica particles).

### Methods of Preparation

A process for making the semi-permeable particles involves basically a multi-step process. A first aqueous phase (primarily water as a solvent) is formed having dispersed therein the nanoparticles of catalytically active metallic materials and dissolved therein, one or more cavity stabilizing hydrocolloids (both described above). While not intending to be limiting for this invention, the nanoparticles of catalytically active metallic materials can be present in this first aqueous phase such that the amount of nanoparticles in the semi-permeable particles can be at least 1 part per million and up to and including 20 weight % solids, or typically at least 10 parts per million and up to and including 10 weight %, based on the total dry weight of the semi-permeable particles. The one or more cavity stabilizing hydrocolloids can be present in this first aqueous phase in an amount of at least 0.1 weight % and up to and including 20 weight %, or typically of at least 0.5 weight % and up to and including 10 weight %, all based on the total first aqueous phase weight.

This first aqueous phase is then dispersed in a suitable organic solution (one or more organic solvents described below) or oil phase comprising one or more of the water-insoluble semi-permeable polymers (described above) that eventually form a continuous semi-permeable polymeric phase, to form a first emulsion (first water-in-oil emulsion). These water-insoluble polymers are dissolved in the organic solvent. The first aqueous phase creates the discrete cavities in the resulting semi-permeable particles. Ways to form the first emulsion are described below.

Salts can be added to the first aqueous phase to buffer the emulsion and optionally to control the osmotic pressure of the aqueous phases. When CMC is used as a cavity stabilizing hydrocolloid, for example, the osmotic pressure can be increased by using inorganic salts or a pH 7 buffer. The first emulsion can also contain additional cavity forming agents such as ammonium carbonate.

The semi-permeable particles can be prepared and provided in dry powder form or as an aqueous slurry. They can be used in either form.

Any suitable organic solvent that will dissolve the water-insoluble, semi-permeable polymer(s) and that is also immiscible with water can be used to prepare the organic solvent used in the first emulsion. Such organic solvents include but are not limited to, ethyl acetate, propyl acetate, chloromethane, dichloromethane, vinyl chloride, trichloromethane, carbon tetrachloride, ethylene chloride, trichloroethane, toluene, xylene, cyclohexanone, 2-nitropropane, dimethyl carbonate, and mixtures of two or more of these solvents. Ethyl acetate and propyl acetate are generally good solvents for many useful water-insoluble semi-permeable polymers while being sparingly soluble in water, and they are readily removed as described below by evaporation.

Optionally, the organic solution is a mixture of two or more water-immiscible solvents chosen from the list given above. For example, the organic solution can comprise a mixture of one or more of the above organic solvents with a water-immiscible non-solvent for the water-insoluble semi-permeable polymer such as heptane, cyclohexane, and diethylether that is added in a proportion that is insufficient to precipitate the water-insoluble semi-permeable polymer prior to drying and isolation.

Depending upon the ultimate use of the semi-permeable particles, the first emulsion can also include various additives, generally that are added to the water-insoluble semi-permeable polymer prior to its dissolution in the organic solvent, during dissolution, or after the dissolution step itself. Such additives can include but are not limited to, colorants, charge control agents, shape control agents, compatibilizers, wetting agents, surfactants, plasticizers, and release agents such as waxes and lubricants, that are not within the cavities. Combinations of these materials can also be used. The first or second aqueous phase can also include a buffering salt examples of which are readily known in the art.

The next step in the formation of porous particles according to this invention involves forming a water-in-oil-in-water emulsion by dispersing the first emulsion (first water-in-oil emulsion) in a second aqueous phase containing a surface stabilizing material to form a second emulsion (water-in-oil-in-water emulsion) that contains droplets of the first water-in-oil emulsion. The surface stabilizing materials can be either stabilizer polymers such as poly(vinyl pyrrolidone) or poly(vinyl alcohol) or more likely a colloidal silica such as that available as LUDOX® or NALCO® silica or latex particles in a modified ELC process such as described in U.S. Pat. No. 4,965,131 (Nair et al.), U.S. Pat. No. 2,934,530 (Ballast et al.), U.S. Pat. No. 3,615,972 (Cohrs et al.), U.S. Pat. No. 2,932,629 (Wiley), and U.S. Pat. No. 4,314,932 (Wakimoto et al.), all of which are incorporated herein by reference.

The second aqueous phase comprises primarily water as the solvent, and it can also comprise buffering salts, shape control agents, surface stabilizing materials, and co-stabilizers or promoters to drive the surface stabilizing materials, particularly colloidal material, to the interface of the water-in-oil droplets in the second aqueous phase.

Suitable co-stabilizers or promoters include sulfonated polystyrenes, alginates, derivatives of cellulose, tetramethyl ammonium hydroxide or chloride, triethylphenyl ammonium hydroxide, triethylphenyl ammonium hydroxide, triethylphenyl ammonium chloride, diethylaminoethylmethacrylate, water-soluble complex resinous amine condensation products, such as the water soluble condensation product of diethanol amine and adipic acid, such as poly(adipic acid-co-methylaminoethanol), water soluble condensation products of ethylene oxide, urea, and formaldehyde and polyethyleneimine; gelatin, glue, casein, albumin, gluten, and the like. A particularly useful promoter is poly(adipic acid-co-methylaminoethanol). The amount of any of the co-stabilizers or promoters used in the present invention can be at least 0.1 weight % to and including 20 weight % based on the total dry weight of the surface stabilizing materials.

The first emulsion used to prepare the semi-permeable particles of this invention can be prepared by any known emulsifying technique and conditions using any type of mixing and shearing equipment. Such equipment includes but is not limited to, a batch mixer, planetary mixer, single or multiple screw extruder, dynamic or static mixer, colloid mill, high pressure homogenizer, sonicator, or a combination thereof. While any high shear type agitation device is useful, a particularly useful homogenizing device is the Microfluidizer® such as Model No. 110T produced by Microfluidics Manufacturing operating at >5000 psi. In this device, the droplets of the first aqueous phase can be dispersed and reduced in size in the organic solution in a high flow agitation zone and, upon exiting this zone, the size of the droplets in the dispersed aqueous phase is reduced to uniform sized dispersed droplets in the organic solution. The temperature of the process can be modified to achieve the optimum viscosity for emulsification of the droplets and to minimize evaporation of the organic solution.

Specifically, the water-in-oil emulsion is mixed with the second aqueous phase containing a surface stabilizing material such as colloidal silica and an optional co-stabilizer, to form an aqueous suspension of droplets of the water-in-oil emulsion in the second aqueous phase, which is then subjected to shear or extensional mixing or shear flow processes, such as through an orifice device to reduce the droplet size of the suspension, yet greater than the particle size of the first water-in-oil emulsion, to achieve narrow size distribution droplets through the limited coalescence process. The pH of the second aqueous phase is generally between 4 and 7 when silica particles are used as the surface stabilizing material. Useful surface stabilizing materials and co-stabilizers or promoters are described above. Colloidal or fused silica (for example LUDOX™ or NALCO™) is particularly useful. The actual amount of surface stabilizing material used depends upon the final desired size of the porous particles, which in turn depends upon the volume and weight ratios of the various phases used for making the multiple emulsions. While not intending to be limiting for this invention, the amount of surface stabilizing material in the second emulsion can be at least 0.1 weight % and up to and including 10 weight %, or typically at least 0.2 weight % and up to and including 7 weight %, based on the total weight of the water-in-oil phase in the water-in-oil-in-water emulsion and depending upon the particle size of the surface stabilizing material.

Specifically, the water-in-oil emulsion is mixed with the second aqueous phase containing colloidal silica stabilizer and an optional co-stabilizer, to form an aqueous suspension of droplets of the water-in-oil emulsion in the second aqueous phase, which is then subjected to shear or extensional mixing or shear flow processes, such as through an orifice device to reduce the droplet size of the suspension, yet greater than the particle size of the first water-in-oil emulsion, to achieve narrow size distribution droplets through the limited coalescence process. The pH of the second aqueous phase is generally between 4 and 7 when silica particles are used as the colloidal stabilizer. Useful surface stabilizing materials and co-stabilizers or promoters are described above. Colloidal or fused silica (for example LUDOX™ or NALCO™) is particularly useful. The actual amount of surface stabilizing material used depends upon the final desired size of the porous particles, which in turn depends upon the volume and weight ratios of the various phases used for making the multiple emulsions. While not intending to be limiting for this invention, the amount of surface stabilizing material in the second emulsion can be at least 0.1 weight % and up to and including 10 weight %, or typically at least 0.2 weight % and up to and including 7 weight %, based on the total weight of the water-in-oil phase in the water-in-oil-in-water emulsion and depending upon the particle size of the surface stabilizing material.

When the second (water-in-oil-in-water) emulsion is formed, shear or extensional mixing or flow process is controlled in order to minimize disruption of the distinct droplets of the first aqueous phase in the organic solution. Droplet size reduction is achieved by homogenizing the second emulsion through a capillary orifice device, or other suitable flow geometry. The shear field used to create the droplets can be created using standard shear geometries, such as an orifice plate or capillary. However, the flow field can also be generated using alternative geometries, such as packed beds of beads, or stacks or screens that impart an additional extensional component to the flow. It is well known in the literature that membrane-based emulsifiers can be used to generate multiple emulsions. The techniques allow the droplet size to be tailored across a wider range of sizes by adjusting the cavity volume or mesh size, and can be applied across a wide range of flow rates. The back pressure suitable for producing acceptable particle size and size distribution is at least 100 psi (689.5 kilonewtons/m²) and up to and including 5000 psi (34,475 kilonewtons/m²), or typically at least 500 psi (3447.5 kilonewtons/m²) and up to and including 3000 psi (20,685 kilonewtons/m²). The flow rate is generally at least 1000 ml/min and up to and including 6000 ml/min, particularly when a capillary orifice device is used.

The final size of the semi-permeable particles and the final size of the multiple discrete cavities of the semi-permeable particles can be impacted by the osmotic mismatch between the osmotic pressure of the first and second aqueous phases. At each interface, the larger the osmotic pressure gradient present, the faster the diffusion rate where water will diffuse from the lower osmotic pressure phase to the higher osmotic pressure phase depending on the solubility and diffusion coefficient in the organic solution.

The organic solution is removed after the first emulsion droplets are formed in the second aqueous phase. Removal of the organic solvents provides precursor semi-permeable particles that can be subjected to isolation from the second aqueous phase, washing, and optional drying techniques to provide the desired semi-permeable particles. The details of these procedures depend upon the water solubility and boiling points of the organic solvents in the organic solution relative to the temperature of the solvent removal process. Generally, organic solvents can be removed by evaporation using removal apparatus such as a rotary evaporator or a flash evaporator. The semi-permeable particles can then be isolated after removing the organic solvents by filtration or centrifugation, washing to remove any contamination from the second aqueous phase, optionally followed by drying, for example, in an oven at 40° C. that also removes any water remaining in the discrete cavities. Advantageously, the semi-permeable particles can be used directly without removing the water from the discrete cavities, that is as an aqueous slurry. Optionally, the semi-permeable particles can be treated with alkali to remove any surface stabilizing material if desired.

Optionally, after the second emulsion has been formed, additional water can be added to the second emulsion (water-in-oil-in-water emulsion) to increase the size of the multiple discrete cavities by creating an osmotic pressure mismatch between the first and second aqueous phases allowing for the migration of water from the second aqueous phase to the first.

Alternatively, in the method for preparing the semi-permeable particles of the invention, the organic solution described above can be replaced with one or more ethylenically unsaturated polymerizable monomers (generally in liquid form) and a polymerization initiator to form a second emulsion (water-in-oil-in-water emulsion). Thus, the organic solution comprises predominantly the ethylenically unsaturated polymerizable monomers as the organic solvents. The ethylenically unsaturated polymerizable monomers in the second emulsion can be polymerized for example through the application of heat or radiation (such as actinic or IR radiation) after the second emulsion is formed and before any organic solvents are removed to form one or more suitable water-insoluble semi-permeable polymers. Any organic solvents can be present in such small amounts and have sufficient solubility in water that it can be removed by washing with water. This washing can occur simultaneously with a filtration process. The resulting suspension of polymerized precursor semi-permeable particles can be isolated and re-slurried in water as described earlier to yield semi-permeable particles of this invention.

In addition, if desired, the water-immiscible ethylenically unsaturated polymerizable monomer(s) can be used in mixture with one or more water-insoluble, semi-permeable polymers as described above. Useful ethylenically unsaturated polymerizable monomers and polymerization initiators would be readily apparent to one skilled in the art in order to achieve the desired continuous polymeric phase.

The shape of the semi-permeable particles can be modified if necessary by reducing the spherical nature (sphericity) of the particles (for example, an aspect ratio of less than 0.95, or an aspect ratio of from 0.4 and up to and including 0.95). In the method used to prepare the semi-permeable particles, additives (shape control agents) can be incorporated into the first aqueous phase or in the organic solution to modify the shape, aspect ratio, or morphology of the resulting semi-permeable particles. The shape control agents can be added after or prior to forming the second emulsion. Some useful shape control agents are quaternary ammonium tetraphenylborate salts described in U.S. Patent Application Publication 2007/0298346 (Ezenyilimba et al.), metal salts described in U.S. Patent Application Publication 2008/0145780 (Yang et al.), carnauba waxes described in U.S. Pat. No. 5,283,151 (Santilli), SOLSPERSE® hyperdispersants as described in U.S. Pat. No. 5,968,702 (Ezenyilimba et al.), metal salts as described in U.S. Pat. No. 7,655,375 (Yang et al.), and zinc organic complexes as described in U.S. Pat. No. 7,662,535 (Yang et al.). All of these publications are incorporated herein by reference. The more desirable shape control agents are polyethyloxazoline, fatty acid modified polyesters such as EFKA® 6225 and EFKA° 6220 from Ciba BASF, and phosphate esters of alkoxylated phenols such as SynFac® 8337.

The method for causing a chemical reaction according to this invention can be carried out by contacting a reactive chemical having a molar mass of 1000 Daltons or less with a slurry of semi-permeable particles of this invention. This contact can be achieved by stirring or otherwise agitating a slurry of the metallic catalyst-containing semi-permeable particles with a substantially aqueous solution of a suspension of the reactive chemical in a vessel for the required period of time, maintaining the temperature of the mixture as desired by any conventional means such as a thermostated jacket around the vessel. In a second embodiment, a column can be packed with the metallic catalyst-containing semi-permeable particles, and a substantially aqueous solution of a suspension of the reactive chemical can be allowed to flow through the stationary “bed” of semi-permeable particles to affect the desired chemical reaction. The temperature of the column can be maintained by conventional means to achieve a desired reaction rate.

The present invention provides at least the following embodiments and combinations thereof, but other combinations of features are considered to be within the present invention as a skilled artisan would appreciate from the teaching of this disclosure:

1. A semi-permeable particle comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the multiple discrete cavities, the semi-permeable particle being permeable to molecules having a molar mass of 1000 Daltons or less,

wherein the semi-permeable particle has a mode particle size of at least 1 μm and comprises nanoparticles of catalytically active metallic materials disposed within at least some of the multiple discrete cavities,

which nanoparticles of catalytically active metallic materials (a) comprise one or more elements selected from Groups 8, 9, 10, and 11 of the Periodic Table, and (b) have an effective diameter of at least 1 nm and up to and including 200 nm.

2. The semi-permeable particle of embodiment 1, wherein the water-insoluble semi-permeable polymer is selected from a polyester, polyamide, polyurethane, styrenic polymer, mono-olefin polymer, vinyl ester polymer, acrylic polymer, vinyl ether polymer, vinyl ketone polymer, and aliphatic cellulose ester polymer.

3. The semi-permeable particle of embodiment 1 or 2, comprising a cavity stabilizing hydrocolloid that is selected from the group consisting of carboxymethyl cellulose (CMC), a gelatin or gelatin derivative, a protein or protein derivative, a hydrophilic synthetic polymer, a water-soluble microgel, a polystyrene sulfonate, poly(2-acrylamido-2-methylpropanesulfonate), and a polyphosphate.

4. The semi-permeable particle of any of embodiments 1 to 3 that has a mode particle size of at least 1 μm and up to and including 100 μm.

5. The semi-permeable particle of any embodiments 1 to 4, wherein each semi-permeable particle further comprises an amphiphilic (low HLB) block copolymer that is disposed at the interface of the multiple discrete cavities and the continuous polymeric phase.

6. The semi-permeable particle of any of embodiments 1 to 5, wherein the volume of the multiple discrete cavities is at least 10% and up to and including 60% of the total dry semi-permeable particle volume.

7. The semi-permeable particle of any of embodiment 1 to 6, wherein the average discrete cavity size is at least 100 nm to and including 5 μm.

8. The semi-permeable particle of any of embodiments 1 to 7 having an aspect ratio of at least 0.4.

9. The semi-permeable particle of any of embodiments 1 to 8 further comprising a surface stabilizing material on the external particle surface. 10. The semi-permeable particle of any of embodiments 1 to 9 further comprising colloidal or fumed silica on the external particle surface.

11. The semi-permeable particle of any of embodiments 1 to 10 comprising nanoparticles of catalytically active metallic materials comprising palladium, platinum, or nickel in the multiple discrete cavities, the nanoparticles having an effective diameter of at least 2 nm and up to and including 100 nm.

12. The semi-permeable particle of any of embodiments 1 to 11, wherein the nanoparticles of catalytically active metallic materials have an effective diameter of at least 2 nm and up to and including 50 nm.

13. An aqueous slurry of multiple semi-permeable particles according to any of embodiments 1 to 12.

14. A method of making an aqueous dispersion of a plurality of semi-permeable particles of any of embodiments 1 to 12, each semi-permeable particle further comprising multiple discrete cavities within the continuous polymeric phase, and a cavity stabilizing hydrocolloid disposed within at least some of the multiple discrete cavities, the semi-permeable particle being permeable to molecules having a molar mass of 1000 Daltons or less,

wherein the semi-permeable particle has a mode particle size of at least 1 μm and comprises nanoparticles of catalytically active metallic materials disposed within at least some of the multiple discrete cavities,

which nanoparticles of catalytically active metallic materials (a) comprise one or more elements selected from Groups 8, 9, 10, and 11 of the Periodic Table, and (b) have an effective diameter of at least 1 nm and up to and including 200 nm,
the method comprising:

providing a first aqueous phase comprising the nanoparticles of catalytically active metallic materials and the cavity stabilizing hydrocolloid, both dispersed within the first aqueous phase,

dispersing the first aqueous phase in an organic solvent comprising a water-insoluble polymer to form a first emulsion,

dispersing the first aqueous phase in an organic solvent comprising the water-insoluble semi-permeable polymer to form a first water-in-oil emulsion,

dispersing the first water-in-oil emulsion in a second aqueous phase containing a surface stabilizing material to form a water-in-oil-in-water emulsion containing droplets of the water-in-oil emulsion, and

removing the organic solvent from the droplets to form the aqueous dispersion of a plurality of semi-permeable particles.

15. A method for causing a chemical reaction, comprising:

contacting one or more reactive chemicals having a molar mass of 1000 Daltons or less with a slurry of semi-permeable particles as described in any of embodiments 1 to 11,
each of the semi-permeable particles comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the multiple discrete cavities, the semi-permeable particle being permeable to the one or more reactive chemicals having a molar mass of 1000 Daltons or less,
wherein the semi-permeable particle has a mode particle size of at least 1 μm and comprises nanoparticles of catalytically active metallic materials disposed within at least some of the discrete cavities, the catalytically active metallic materials being capable of catalyzing a chemical conversion of the one or more reactive chemicals having a molar mass of 1000 Daltons or less,
which nanoparticles of catalytically active metallic materials (a) comprise one or more elements selected from Groups 8, 9, 10, and 11 of the Periodic Table, and (b) have an effective diameter of at least 1 nm and up to and including 200 nm.

The following Examples are provided to illustrate the practice of this invention and are not meant to be limiting in any manner.

Synthesis of Palladium Nanoparticles:

A mixture of 300 ml of 2.0 mmolar H₂PdCl₄ solution, 420 ml of deionized water, 1.334 g of poly(vinyl pyrrolidone) (˜20:1 monomer:Pd), and 80 drops of 1.0 molar aqueous HCl was heated to reflux. This mixture was removed from heat and 280 ml of ethanol was added. The reaction mixture was returned to reflux, held for 3 hours, cooled to ambient temperature, neutralized with NaOH, and concentrated at reduced pressure. The resulting nanoparticles were isolated by repeated re-suspension in 50% aqueous acetone, followed by centrifugation at 3000 rpm for 30 minutes. The product was isolated as 0.7332 g of a brown powder.

The amphiphilic block copolymer of polyethylene oxide and polycaprolactone (PEO-b-PCL) was prepared using the procedure described in U.S. Pat. No. 5,429,826 (Nair et al.) and was designed to have the following molecular weights in the block components where the first number is the molecular weight of the hydrophilic block segment and the second number is the molecular weight of the oleophilic block segment: 5,000 and 25,000.

### INVENTION EXAMPLE 1

### Preparation of Semi-Permeable Particles Containing Nanoparticles of Palladium

A first aqueous phase (W1) was prepared using 37.4 g of a 3 weight % of a carboxy methyl cellulose sodium salt solution in water along with 19.8 g of a 0.006 weight % of palladium nanoparticles solution in water. An oil phase was made using 52.7 g of a 23.2 weight % solution of polycaprolactone (MW 45,000) obtained from Sigma Aldrich Company in ethyl acetate, 11.1 g of a 23.5 weight % solution of poly(ethylene oxide-b-caprolactone) in ethyl acetate, and 122.3 g of ethyl acetate. The aqueous phase (W1) was added to the oil phase (O) followed by mixing using a Silverson L4R Mixer fitted with a small holed disintegrating head. The resulting water-in-oil (W1/O) first emulsion was homogenized by using a Microfluidizer model 110T from Microfluidics at a pressure of 8000 psi. This homogenized first emulsion was added to a second aqueous phase (W2) comprising 12.7 g of a 50 weight % solution of Nalco 1060 colloidal silica in water, 387.6 g of a pH 4 citrate/phosphate buffer, and 6.3 g of a 10 weight % solution of poly(methyl amino ethanol) adipate (co-stabilizer, prepared using known procedures and starting materials) in water.

The resulting second emulsion was stirred using a Silverson L4R Mixer fitted with a large holed disintegrating head. The ethyl acetate was evaporated from the second emulsion using a Buchi ROTA VAPOR RE120 evaporator at 40° C. under reduced pressure to yield precursor semi-permeable particles containing palladium nanoparticles in the resulting cavities. The precursor semi-permeable particles were washed on a glass frit funnel, and stored as a suspension of semi-permeable particles in distilled water. The resulting particles were broadly distributed with a mean size of 3.2 μm and a coefficient of variation of 90. Elemental analysis determined that the concentration of palladium was 53 μg per g of semi-permeable particles (dry basis).

### INVENTION EXAMPLE 2

### Preparation of Pd-Loaded Semi-Permeable Particle Slurry (High Concentration of Pd Nanoparticles)

The procedure of Invention Example 1 was repeated except that the first aqueous phase (W1) was made using 0.15 weight % of Pd nanoparticles solution in water. Elemental analysis determined that the concentration of Pd in the resulting semi-permeable particles was 1300 μg per g of semi-permeable particles (dry basis).

### INVENTION EXAMPLE 3

### Preparation of Pd-Loaded Semi-Permeable Particle Slurry (Narrow Size Distribution Particles)

The procedure of Invention Example 1 was repeated except that the W1 phase was made using a 0.12 weight % Pd nanoparticle solution in water. The particles were run through an orifice homogenizer before evaporation of the ethyl acetate, which produced semi-permeable particles having a narrow size distribution. The resulting particles were narrowly distributed with a mean size of 3.6 μm and a coefficient of variation of 50. Elemental analysis determined that the concentration of Pd was 500 μg per g of semi-permeable particles (dry basis).

### INVENTION EXAMPLE 4

### Hydrogenation of 2-Butyne-1,4-diol Using Catalytic Pd Nanoparticle Loaded Semi-Permeable Particles

A sample of the 2-butyne-1,4-diol reactant (1.0 g, 12 mmol) was dissolved in a mixture of 15 ml of water and 10 ml of the aqueous Pd semi-permeable particle suspension described in Invention Example 1 in a heavy-walled glass bottle. The Pd:reactant ratio was approximately 10 ppm. Hydrogen was introduced at 48 psi (330 kilonewtons/m²) and the mixture was shaken continuously at ambient temperature. Periodically, small aliquots were removed for analysis by gas chromatography (GC). After each sample was taken, the mixture was re-pressurized with hydrogen, and the reaction was continued. Samples for GC were filtered through 0.45 mm PVDF membranes before injection. The concentrations of the reactant and the resulting hydrogenation product, 2-butene-1,4-diol, were determined by GC as follows:

| Time (minutes) | [2-Butyne-1,4-diol] | [2-Butene-1,4-diol] |
|---|---|---|
| 0 | 100% | 0% |
| 60 | 97.5% | 2.5% |
| 138 | 95.0% | 4.9% |
| 192 | 93.0% | 6.8% |
| 246 | 90.6% | 9.2% |

These results show that the Pd nanoparticle loaded semi-permeable particles of the present invention can be used effectively for the catalytic reaction (hydrogenation) of 2-butyne-1,4-diol.

### INVENTION EXAMPLE 5

### Hydrogenation of 2-Butyne-1,4-diol Using Catalytic Pd Nanoparticle Loaded Semi-permeable Particles (Effect of Increased Pd Content)

The procedure of Invention Example 4 was followed, except using the aqueous Pd semi-permeable particle suspension of Invention Example 2. The Pd:reactant ratio was approximately 255 ppm. In this experiment, the second resulting hydrogenation product, 1,4-butanediol, was also detected as well as the first product. The concentrations of the reactant and two products were determined by GC as follows:

| Time | [2-Butyne-1,4- | | |
|---|---|---|---|
| (minutes) | diol] | [2-Butene-1,4-diol] | [1,4-Butanediol] |
| 0 | 100% | 0% | 0% |
| 60 | 73.6% | 26.0% | 0.3% |
| 138 | 33.3% | 65.2% | 1.1% |
| 192 | 0.2% | 33.9% | 49.4% |
| 246 | 0.2% | 29.4% | 53.6% |

These results show that 2-butyne-1,4-diol was successfully hydrogenated using the semi-permeable particles of this invention containing the catalytic Pd nanoparticles, and that the increased concentration of the Pd nanoparticles led to more rapid and extensive hydrogenation.

### INVENTION EXAMPLE 6

### Hydrogenation of 2-Butyne-1,4-diol Using Catalytic Pd Nanoparticles in Semi-permeable Particles (Effect of Narrow Size Distribution Particles)

The procedure of Invention Example 4 was followed, except using the aqueous Pd microparticle suspension of Invention Example 3. The Pd:reactant ratio was approximately 81 ppm. In this experiment, the second resulting hydrogenation product, 1,4-butanediol, was also detected as well as the first product. The concentrations of the reactant and two products were determined by GC as follows:

| Time | [2-Butyne-1,4- | | |
|---|---|---|---|
| (minutes) | diol] | [2-Butene-1,4-diol] | [1,4-Butanediol] |
| 0 | 100% | 0% | 0% |
| 64 | 60.9% | 38.4% | 0.5% |
| 126 | 19.9% | 78.2% | 1.3% |
| 194 | 0.1% | 67.3% | 26.8% |
| 259 | 0.1% | 39.5% | 49.9% |
| 318 | 0% | 23.0% | 63.6% |

These results show that 2-butyne-1,4-diol was successfully hydrogenated using the semi-permeable particles of this invention containing the catalytic Pd nanoparticles, and that the more uniform size distribution particles led to rapid hydrogenation.

### INVENTION EXAMPLE 7

### Hydrogenation of 2-Hydroxyethyl Acrylate Using Pd Nanoparticle Loaded Semi-permeable Particles

The procedure of Invention Example 4 was followed, except 2-hydroxyethyl acrylate was used instead of 2-butyne-1,4-diol. The Pd:reactant ratio was approximately 28 ppm. The concentrations of the reactant, 2-hydroxyethyl acrylate, and resulting product, 2-hydroxyethyl propionate, were determined by GC as follows:

| | | [2-Hydroxyethyl |
|---|---|---|
| Time (minutes) | [2-Hydroxyethyl acrylate] | propionate] |
| 0 | 100% | 0% |
| 60 | 76.9% | 23.1% |
| 122 | 60.4% | 39.6% |
| 182 | 48.1% | 51.9% |

These results show that the catalytic Pd nanoparticle loaded semi-permeable particles of this invention are also capable of catalyzing the hydrogenation of the reactant, 2-hydroxyethyl acrylate.

### INVENTION EXAMPLE 8

### Hydrogenation of 2-Hydroxyethyl Acrylate Using Pd Nanoparticles in Semi-permeable Particles (Effect of Increased Pd Content)

The procedure of Invention Example 7 was followed, except using the aqueous Pd semi-permeable particle suspension of Invention Example 2. The Pd:reactant ratio was approximately 170 ppm. The concentrations of the reactant, 2-hydroxyethyl acrylate, and resulting product, 2-hydroxyethyl propionate, were determined by GC as follows:

| | | [2-Hydroxyethyl |
|---|---|---|
| Time (minutes) | [2-Hydroxyethyl acrylate] | propionate] |
| 0 | 100% | 0% |
| 60 | 0.1% | 99.9% |

These results show that 2-hydroxyethyl acrylate was successfully hydrogenated using the catalytic Pd nanoparticles in the semi-permeable particles, and that the increased concentration of the Pd nanoparticles provided more rapid hydrogenation.

### INVENTION EXAMPLE 9

### Recycling and Reuse of Catalytic Pd Nanoparticle Loaded Semi-Permeable Particles

The procedure of Invention Example 4 was followed. After hydrogenation had been run for approximately 20 hours, the reaction mixture was centrifuged at 5000 rpm for 10 minutes to sediment the semi-permeable particles. The supernatant was discarded, and the semi-permeable particles were re-suspended in distilled water. The process was repeated four times, at which point no significant signals in the supernatant could be detected by GC. A weighed aliquot of the final suspension was dried for 24 hours in a vacuum oven at 80° C. and re-weighed to determine the % solids. The final suspension was transferred to a hydrogenation bottle, and amounts of 2-butyne-1,4-diol and water were added to approximate the Pd:reactant ratio and initial concentration of the first hydrogenation. A second hydrogenation using these microparticles was carried out as described in Invention Example 4. Then, the preceding steps of isolating, washing and re-suspending the semi-permeable particles was repeated, and a third hydrogenation was carried out as described in Invention Example 4 was carried out. In these three experiments, the Pd:reactant ratio was approximately 21 ppm. The concentration of the reactant after each cycle was determined by GC as follows:

| | First Cycle | Second Cycle | Third Cycle |
|---|---|---|---|
| | [2-Butyne-1,4- | [2-Butyne-1,4- | [2-Butyne-1,4- |
| Time (minutes) | diol] | diol] | diol] |
| 0 | 100% | 100% | 100% |
| 60 | 95.0% | 75.4% | 67.5% |
| 120 | 90.0% | 47.7% | 31.3% |
| 180 | 84.4% | 15.8% | 0.04% |
| 240 | 78.4% | 0.4% | 0.3% |
| 300 | 72.3% | 0.4% | 0.3% |

The expected amounts of the hydrogenation products, 2-butene-1,4-diol and 1,4-butanediol were also detected in these experiments. These results show that the catalytic Pd nanoparticle loaded semi-permeable particles of this invention can be used repeatedly for the hydrogenation of 2-butene-1,4-diol, and that the catalytic activity increased as the semi-permeable particles were reused.

### COMPARATIVE EXAMPLE 1

### Hydrogenation of 2-Butene-1,4-diol Using Non-Entrapped Pd Nanoparticles

The procedure of Invention Example 4 was followed, except using the non-encapsulated suspension of Pd nanoparticles described above in Invention Example 1 (the Pd nanoparticles were not in cavities of the polymeric particles). The Pd:reactant ratio was approximately 21 ppm. The concentrations of the reactant, 2-butyne-1,4-diol, and the two resulting products were determined by GC as follows:

| Time | [2-Butyne-1,4- | | |
|---|---|---|---|
| (minutes) | diol] | [2-Butene-1,4-diol] | [1,4-Butanediol] |
| 0 | 100% | 0% | 0% |
| 60 | 87.6% | 12.2% | 0.2% |
| 120 | 76.6% | 22.9% | 0.3% |
| 180 | 65.0% | 34.4% | 0.4% |
| 240 | 51.9% | 47.3% | 0.6% |
| 300 | 37.2% | 61.7% | 0.7% |
| 1260 | 0.1% | 69.7% | 25.4% |

These results taken together with those provided in Invention Example 4 show that the hydrogenation of 2-butyne-1,4-diol using catalytic Pd nanoparticles contained within the cavities of the semi-permeable particles proceeded at a sufficiently substantial rate compared to the same reaction using Pd nanoparticles outside the semi-permeable particles.

COMPARATIVE EXAMPLE 2

### Hydrogenation of 2-Butene-1,4-diol Using Semi-Permeable Particles Without Catalytically Active Metallic Materials

The procedure of Invention Example 4 was followed using similar semi-permeable particles, except no catalytically active metallic materials were used. No hydrogenation products were observed and the reactant was recovered unchanged. These results show that the catalytically active metallic materials described herein are required to cause the desired chemical reactions. Empty semi-permeable particles were ineffective.

### INVENTION EXAMPLE 10

### Hydrogen Peroxide Decomposition Catalyzed Using Pd Nanoparticle Loaded Semi-Permeable Particles

Catalytic Pd nanoparticles loaded in semi-permeable particles of this invention in a slurry (0.03 ml) according to Invention Example 2 was added to 3.0 ml of a 0.0610 molar hydrogen peroxide solution. The dispersion was stirred and analyzed periodically for hydrogen peroxide concentration by filtering aliquots through a 0.45 μm UNIPREP glass microfiber filter and measuring absorbance at 240 nm (hydrogen peroxide characteristic absorption peak). The absorbance was converted to a concentration using known titration of hydrogen peroxide by KMnO₄ in acidic solution. A concentration calibration curve was constructed by dilution of the titrated sample. A control sample of hydrogen peroxide solution without catalytic Pd nanoparticles in semi-permeable particles did not show any change in absorption at 240 nm under the same conditions. The results are as follows:

| Time (minutes) | [Hydrogen Peroxide] (mol/l) |
|---|---|
| 5 | 0.0369 |
| 15 | 0.0364 |
| 30 | 0.0342 |
| 60 | 0.0328 |
| 90 | 0.0276 |

These results show that the Pd nanoparticle loaded semi-permeable particles successfully catalyzed the decomposition of hydrogen peroxide according to the present invention.

The invention has been described in detail with particular reference to certain preferred embodiments thereof, but it will be understood that variations and modifications can be effected within the spirit and scope of the invention.

1. A semi-permeable particle comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the multiple discrete cavities, the semi-permeable particle being permeable to molecules having a molar mass of 1000 Daltons or less,
wherein the semi-permeable particle has a mode particle size of at least 1 μm and comprises nanoparticles of catalytically active metallic materials disposed within at least some of the multiple discrete cavities,
which nanoparticles of catalytically active metallic materials (a) comprise one or more elements selected from Groups 8, 9, 10, and 11 of the Periodic Table, and (b) have an effective diameter of at least 1 nm and up to and including 200 nm.

2. The semi-permeable particle of claim 1, wherein the water-insoluble semi-permeable polymer is selected from a polyester, polyamide, polyurethane, styrenic polymer, mono-olefin polymer, vinyl ester polymer, acrylic polymer, vinyl ether polymer, vinyl ketone polymer, and aliphatic cellulose ester polymer.

3. The semi-permeable particle of claim 1, comprising a cavity stabilizing hydrocolloid that is selected from the group consisting of carboxymethyl cellulose (CMC), a gelatin or gelatin derivative, a protein or protein derivative, a hydrophilic synthetic polymer, a water-soluble microgel, a polystyrene sulfonate, poly(2-acrylamido-2-methylpropanesulfonate), and a polyphosphate.

4. The semi-permeable particle of claim 1, wherein each semi-permeable particle further comprises an amphiphilic (low HLB) block copolymer that is disposed at the interface of the multiple discrete cavities and the continuous polymeric phase.

5. The semi-permeable particle of claim 1 that has a mode particle size of at least 1 μm and up to and including 100 μm.

6. The semi-permeable particle of claim 1, wherein the volume of the multiple discrete cavities is at least 10% and up to and including 60% of the total dry semi-permeable particle volume.

7. The semi-permeable particle of claim 1, wherein the average discrete cavity size is at least 100 nm to and including 5 μm.

8. The semi-permeable particle of claim 1 having an aspect ratio of at least 0.4.

9. The semi-permeable particle of claim 1 further comprising a surface stabilizing material on the external particle surface.

10. The semi-permeable particle of claim 1 further comprising colloidal or fumed silica on the external particle surface.

11. The semi-permeable particle of claim 1 comprising nanoparticles of catalytically active metallic materials comprising palladium, platinum, or nickel in the multiple discrete cavities, the nanoparticles having an effective diameter of at least 2 nm and up to and including 100 nm.

12. The semi-permeable particle of claim 1, wherein the nanoparticles of catalytically active metallic materials have an effective diameter of at least 2 nm and up to and including 50 nm.

13. An aqueous slurry of multiple semi-permeable particles according to claim 1.

14. The aqueous slurry of multiple semi-permeable particles of claim 12 wherein each semi-permeable particle comprises one or more nanoparticles of catalytically active metallic materials comprising elements selected from one or more of palladium, platinum, rhodium, ruthenium, nickel, cobalt, iron, copper, silver, gold, iridium, and osmium.

15. A method of making an aqueous dispersion of a plurality of semi-permeable particles, each semi-permeable particle further comprising multiple discrete cavities within the continuous polymeric phase, and a cavity stabilizing hydrocolloid disposed within at least some of the multiple discrete cavities, the semi-permeable particle being permeable to molecules having a molar mass of 1000 Daltons or less,
wherein the semi-permeable particle has a mode particle size of at least 1 μm and comprises nanoparticles of catalytically active metallic materials disposed within at least some of the multiple discrete cavities,
which nanoparticles of catalytically active metallic materials (a) comprise one or more elements selected from Groups 8, 9, 10, and 11 of the Periodic Table, and (b) have an effective diameter of at least 1 nm and up to and including 200 nm,
the method comprising:
providing a first aqueous phase comprising the nanoparticles of catalytically active metallic materials and the cavity stabilizing hydrocolloid, both dispersed within the first aqueous phase,
dispersing the first aqueous phase in an organic solvent comprising a water-insoluble polymer to form a first emulsion,
dispersing the first aqueous phase in an organic solvent comprising the water-insoluble semi-permeable polymer to form a first water-in-oil emulsion,
dispersing the first water-in-oil emulsion in a second aqueous phase containing a surface stabilizing material to form a water-in-oil-in-water emulsion containing droplets of the water-in-oil emulsion, and
removing the organic solvent from the droplets to form the aqueous dispersion of a plurality of semi-permeable particles.

16. The method of claim 15, wherein the water-insoluble semi-permeable polymer in the particles is selected from a polyester, polyamide, polyurethane, styrenic polymer, mono-olefin polymer, vinyl ester polymer, acrylic polymer, vinyl ether polymer, vinyl ketone polymer, and aliphatic cellulose ester polymer.

17. The method of claim 15, wherein the cavity stabilizing hydrocolloid is selected from the group consisting of carboxymethyl cellulose (CMC), a gelatin or gelatin derivative, a protein or protein derivative, a hydrophilic synthetic polymer, a water-soluble microgel, a polystyrene sulfonate, poly(2-acrylamido-2-methylpropanesulfonate), and a polyphosphate.

18. The method of claim 15, wherein each of the plurality of semi-permeable particles has a mode particle size of at least 1 μm and up to and including 100 μm.

19. The method of claim 15, wherein each of the plurality of semi-permeable particles further comprises a surface stabilizing material on the external particle surface.

20. The method of claim 15, wherein each of the plurality of semi-permeable particles further comprises colloidal or fumed silica on the external particle surface.

21. The method of claim 15, wherein each of the plurality of semi-permeable particles comprises nanoparticles of catalytically active metallic materials comprising palladium, platinum, or nickel in the multiple discrete cavities, the nanoparticles having an effective diameter of at least 2 nm and up to and including 100 nm.

22. The method of claim 15, wherein each of the plurality of semi-permeable particles comprises nanoparticles of catalytically active metals that have an effective diameter of at least 2 nm and up to and including 50 nm.

23. A method for causing a chemical reaction, comprising:
contacting one or more reactive chemicals having a molar mass of 1000 Daltons or less with a slurry of semi-permeable particles,
each of the semi-permeable particles comprising a water-insoluble semi-permeable polymer providing a continuous polymeric phase including an external particle surface, the semi-permeable particle further comprising multiple discrete cavities within the continuous polymeric phase, and a cavity stabilizing hydrocolloid disposed within at least some of the multiple discrete cavities, the semi-permeable particle being permeable to the one or more reactive chemicals having a molar mass of 1000 Daltons or less,
wherein the semi-permeable particle has a mode particle size of at least 1 μm and comprises nanoparticles of catalytically active metallic materials disposed within at least some of the multiple discrete cavities, the catalytically active metallic materials being capable of catalyzing a chemical conversion of the one or more reactive chemicals having a molar mass of 1000 Daltons or less,
which nanoparticles of catalytically active metallic materials (a) comprise one or more elements selected from Groups 8, 9, 10, and 11 of the Periodic Table, and (b) have an effective diameter of at least 1 nm and up to and including 200 nm.
